(19) United States
(12) Patent Application Publication
(10) Pub. No.: US 2021/0116345 A1
(43) Pub. Date: Apr. 22, 2021

(54) ONBOARD APPARATUS, SYSTEM, AND METHOD FOR AUTOMATICALLY DYNAMICALLY EVALUATING CHARACTERISTICS OF A NON-HOMOGENOUS LIQUID DURING LOADING AND UNLOADING OF A TRANSPORT CONTAINER

(71) Applicant: Jerry J. Thompson, Harris, MN (US)

(72) Inventor: Jerry J. Thompson, Harris, MN (US)

(21) Appl. No.: 16/462,791

(22) PCT Filed: Nov. 21, 2017
(86) PCT No.: PCT/US17/62876

§ 371 Date: May 21, 2019

Related U.S. Application Data
(60) Provisional application No. 62/425,059, filed on Nov. 21, 2016.

(51) Int. Cl. G01N 9/00; G05D 7/06; G01N 1/20; G01N 33/28

(52) U.S. Cl. CPC G01N 9/00; G05D 7/0617; G01N 2001/1031; G01N 33/2847; G01N 1/20

(57) ABSTRACT

An onboard apparatus, system, and method for automatically loading into or unloading from a bulk transport or other container and evaluating characteristics of a liquid during the loading or unloading, that dynamically monitors and evaluates characteristics of the flow, particularly density, which are used to determine other characteristics and values of the load, namely, presence of contaminants such as water, solids, out of specification conditions, etc. to enable accurately measuring volume, mass, and/or quality of the load, and optionally to automatically responsively perform certain operations, for example, to signal an operator, cease loading, segregate and/or return all or portions of the load, if contaminated or out of specification, and which facilitates control remotely, as well as for qualifying for government certification for custody transfer.

This application is submitted under 35 U.S.C. 371 claiming priority to PCT/US2017/62876, filed Nov. 21, 2017, which application claims the benefit of U.S. Provisional Application No. 62/425,059, filed Nov. 21, 2016.

### TECHNICAL FIELD

The invention relates generally to an onboard apparatus, system, and method for automatically loading into or unloading from a bulk transport container and evaluating characteristics of a liquid during the loading or unloading, and more particularly, that dynamically monitors and evaluates characteristics of the flow, particularly density, which are used to determine other characteristics and values of the load, namely, presence of contaminants such as water, solids, out of specification conditions, etc. to enable accurately measuring volume, mass, and/or quality of the load, and optionally to automatically responsively perform certain operations, for example, to signal an operator, cease loading, segregate and/or return all or portions of the load, if contaminated or out of specification. The invention has particular utility for loading and determining volume and quality of crude oil from collection tanks at remote locations lacking more sophisticated testing and evaluation equipment, including in a manner sufficiently accurately for controlling the loading and unloading remotely, e.g., from a distant control facility, as well as for qualifying for government certification for custody transfer.

### BACKGROUND ART

PCT patent application Ser. No. PCT/US2017/62876, filed Nov. 21, 2017, and U.S. Provisional Application No. 62/425,059, filed Nov. 21, 2016, are incorporated herein by reference in their entirety.

In the oil extraction and processing industry, at larger production and processing volume locations having complex piping networks, it is common to have sophisticated, accurate apparatus for evaluating characteristics of the liquid flows, such as mass flow or density, volumetric flow, viscosity, pressure, temperature, etc. The instruments for measuring these characteristics are regularly maintained, certified, and/or calibrated, and the properties of the liquid flows are generally already known, the instrumentation being used to obtain/monitor precise values for the characteristics, and to signal problems, etc.

In lower production environments, e.g., remote oil production wells, distantly spaced individual well sites, etc., it is not economical to connect the site to a pipe line, or to have in place complex, expensive instrumentation, particularly that requires frequent maintenance, calibration, etc. More commonly, the crude oil is transported by bulk carrier such as a tanker truck or the like from a collection tank or tanks near the production well, to a depot, pipeline terminal, or the like. Generally at these locations, the tanker truck driver or other personnel must manually measure the volume of oil in a stationary tank by a process known as “strapping” which involves lowering a tape measure through a lid or access hatch on the top of the tank and down to the liquid contents prior to loading. In association with this, a sample or samples of the oil are taken at prescribed sampling intervals in the loading process, e.g., ¼; ½; and ¾ through the loading process, to evaluate oil grade, and presence of contaminants such as water, solids, etc., can be noted if desired or required. The present practice is problematic as strapping can involve climbing tall tank ladders, often under inclement weather conditions including ice and snow, and there is a risk of injury or death due to potential deadly gases present when opening the tank hatch. Field measurements of grade and observations of quality can also be less accurate than desired. Measurement of volume by strapping can vary from person to person, and from time to time, resulting in inconsistencies and differences between observed volumes compared to actual volumes measured at a receiving facility, and because strapping is a manual process with no immediate verification, errors and inaccuracies can be difficult to discover.

As another significant issue involved in the transport of oil from smaller producing remote locations, the known manual methods for determining grade and quality of the oil at sampling intervals during loading, while likely accurate for the sampled quantities, will not be accurate for entire load if the grade of the load is not uniform, which is typically the case. In particular in this regard, sampling intervals of ¼; ½; and ¾ through the loading process will not accurately reflect the load where there is a wide range of grades within a tank, and/or wherein contaminants, most importantly, water, gas, salt, and solids (including variously suspended and as sediment) are distributed unevenly within the oil. All of these elements, even though they can be removed to some extent by filters, dehydrators, desalters, de-emulsifiers, and other apparatus at the well head or collection facility if available, can be present to varying extents within a load of crude oil. As an additional factor, vapors and gases some of which are potentially dangerous such as H₂S may be released from the oil when loading and from the transported oil in transit also.

Over time, the oil of the various densities and impurities or contaminants in a collection tank at a remote field location can stratify such that denser or heavier water, alone or containing solids, migrates or settles in the bottom region of the tank, with layers of progressively less dense oil above, and with lighter oils and some solids in the upper region. It is also known for lighter vapors, solids, emulsions, and lighter density oil to be trapped in heavier oils at lower locations of the tank. Stratification can occur at varying rates as a function of a variety of factors, e.g., temperature, flow rate, etc. Accordingly, in one scenario during loading, certain impurities, namely, water and solids, will have settled and can be found mainly in the lowest region of a tank to be unloaded, and thus will be encountered first during the loading operation from the bottom of that tank (See FIG. 12).

During loading, transporting and/or storing crude oil in tanker trucks, gas and vapor can be released, temperature of portions or all of the oil can change, and solids and other contaminants redistributed within the oil as a result of handling and vibration during transport. This presents a different scenario. Under the first scenario, when the contents of the collection tank are loaded from the tank to be unloaded, the contaminants are typically drawn first from the bottom region of the collection tank, and are pumped into the bottom region of the receiving tank wherein they will be agitated and mixed with the rest of the incoming load. Then, depending on the travel conditions, e.g., roughness of the roads, temperature, time, internal convection, etc., the contents of the transport tank upon arrival at an unloading location will be mixed and may be stratified to various extents. Some vapor contents of the load may also have been released. Thereafter, when the transport tank is unloaded, the contents will again typically be drawn from the bottom, resulting in further agitating and mixing. Thus, it can be envisioned that there is virtually no consistency or uniformity with regard to a number of characteristics of crude oil when loaded into a transport container and subsequently unloaded therefrom.

Some characteristics such as viscosity of a particular grade or density of oil will also vary as a result of its temperature. As a result, oil in a region of a tank in direct sunlight and thus having a higher temperature will have a lower viscosity compared to oil of the same grade or density in a shaded region of the tank. Thus, when pumped, the oil from the different regions of the tank will have different flow and mixing characteristics, and thus the distribution of contaminants within a load will not be uniform to any extent.

At some times, a transport tanker may be loaded from different collection tanks without segregation. As a result, contaminants, and vapor and gas losses may not be accurately attributable. It is also possible that not all of a collection tank's contents will be loaded into a transport tank, for any of a variety of reasons. Thus it can be envisioned that there is a demand for safer loading and better data collection, particularly for evaluation of quality and volume, of crude oil received at remote, small production volume sources.

Thus it should be recognized that crude oil is not a routinely uniform consistency and the amount of contaminants present in a particular tank can vary widely for a variety of reasons. Accordingly, the oil quality and value in field collection tanks can differ significantly, including by containing impurities such as water either in liquid state in the bottom of the tank, or an emulsified state, e.g., in mixture with the oil, and other impurities including for example, solids, and salts elsewhere in the tank. Value will also be affected by the composition of the oil itself, e.g., as a function of the various grades or densities that may be present in a particular load. In this regard, as an example, oil within loads transported by truck from the Bakken oil fields of the U.S. have been found to vary in density generally between about 0.7 and 0.8 kg/m³, with solids of lower density and water of higher density contained in various amounts in any give quantity of oil. Some heavier crude oils from other fields will have density values closer to that of water, which will be about 1 kg/m³, but will also vary with contaminant levels such as solids and salts. Thus it should be apparent that manual periodic sampling of grade and observations of impurities, etc., is inadequate for accurately assessing quality and value and provides only a broad estimate. As a result, there is a possibility that a particular load of crude oil will be inaccurately valued in the absence of verifiable data.

When the transported oil is unloaded at the destination, e.g., an oil depot, pipeline terminal, processing facility, etc., which will typically be a larger more complex operation, the crude oil may be better measured and evaluated. However, by this time significant expense may have been incurred in transporting a load over a substantial distance, and at unloading facilities, it is often required to unload quickly to reduce wait times. Thus, it is possible that the more sophisticated measurements will not be taken immediately, such that exact attribution of quality, volume discrepancies, and the like, to a particular load or source may not be possible, giving incentive to some producers to take steps to reduce quality, and some to short or skim from and or dilute loads. If there are quality issues from some producers but not others transported together, it can be difficult to determine the source of the low quality. As a result, a higher quality or more conscientious producer may be penalized by association with a lower quality/less conscientious producer, and the lower quality producer rewarded.

As the value of oil and transport distances increase, the possibility is increased that a load of oil will be tampered with in transit, such as by skimming a portion of the load, and/or by diluting it prior to unloading.

As another issue, most oil fields are not seasonal and the collected oil can be loaded and transported at any time so that temperature and humidity can be a factor in field sampling of characteristics including grade, viscosity, flow rate, water/condensation content, vaporization losses, etc. Oil field equipment such as dehydrators, desalters, filters, separators, etc., can also vary in operational quality and efficiency, calibration, etc., and thus the quality of removal of contaminants from crude oil in the field can vary from load to load. These increase the number of variables that can affect determinations of the value and quality of crude oil collected from remote fields not connected to a piping system.

As noted above, it is known to take metrics of oil flow through pipes of stationary facilities e.g., leased asset custody transfer facilities, which metrics will typically include density, volumetric flow rate, mass flow rate, temperature, pressure, BS&W total (solids and water total), etc., for various purposes, including for evaluating quality, processing baseline values, etc. using various instruments, meters, e.g., process mass flow or density meters, volumetric flow meters, differential pressure meters, and the like. Reference in this regard, meters and related apparatus disclosed and discussed in Sprague U.S. Pat. Nos. 6,957,586 and 7,366,621. However, to maintain desired accuracy, e.g., typically fraction of one percent accuracy of density measurements, the meters must be periodically calibrated using known samples. Being employed at stationary locations facilitates this. Stationary location also eliminates wear and tear, sustained heavy vibrations, and jarring, as would be experienced if the instrumentation were mounted on a mobile platform such as a tanker truck or trailer, railcar, etc., and some instrumentation is too delicate to withstand location on a mobile platform such as a truck or trailer. Still further, it is costly and inconvenient to take mobile platforms out of service for calibration of instrumentation carried thereon.

Portable mass flow or density meters or densitometers such as commercially available Coriolis meters are beginning to be employed to a limited extent on mobile transport tankers for determining total loaded mass or volume and density of crude oil. However, as discussed above, if used for only measuring total loaded volume or mass, there is no differentiation between sources of the loads, and no comprehensive or complete collection of grade or quality data, which represents a lost opportunity for more precisely analyzing and valuing the load especially for custody transfer purposes and for analyzing contaminant content in detail. If more comprehensive data were collected and processed or analyzed at loading, more accurate quality metrics and value could be established at that time and decisions made in regard to oil grade, quality, and value as well as status of the production and collection facility.

Thus, what is sought is a manner of better evaluating properties of bulk liquids, particularly which has the capability to improve accuracy and amount of data collected; reduce physical hazards associated with field measurements; identify and reject substandard loads; detect transit losses, and reduce occurrences of measurement inaccuracies, misunderstandings and other issues and problems associated with loading bulk liquids, particularly crude oil at remote locations such as well sites, oil fields and the like, and which overcomes one or more of the shortcomings and limitations set forth above.

### SUMMARY OF THE INVENTION

What is disclosed is an onboard apparatus, system, and method for automatically loading into or unloading from a bulk transport container and evaluating characteristics of a liquid during the loading or unloading, including in real time or near real time, and more particularly, that improves accuracy and compilation of data collected; and has capabilities including: to automate the measuring process to reduce exposure to physical hazards associated with field activities such as ladder climbing and the like; to identify and flag or reject substandard loads or portions of a load both at the onset of loading and continuously during the loading/unloading; to detect transit losses; and to reduce occurrences of measurement inaccuracies, misunderstandings and other issues and problems associated with loading bulk liquids, particularly crude oil at remote locations such as well sites, oil fields and the like so as to streamline the custody transfer process and provide accurate documentation of load composition and the like, and which overcomes one or more of the shortcomings and limitations set forth above. The invention has particular utility for detecting, quantifying, and optionally segregating water and solids as sediments and emulsions as well as other contaminants and impurities in crude oil loaded from collection tanks at remote locations lacking more sophisticated testing.

According to a preferred aspect of the invention, for process control purposes, the loading of the liquid into a transport container and unloading therefrom can be initiated and/or conducted under manual or automatic control, using conventional apparatus such as, but limited to, conduits such as hoses, pipes, valves, pump or pumps, and piping and hose connections, etc. normally found in a remote oil field or crude oil collection facility. Flow of the liquid can be initiated by gravity and/or pumping. Associated valving is preferably incorporated that is configured and operable to immediately divert and/or direct flow into one or more separate transport compartments or tanks responsively to detection of contaminants, impurities, or out of specification liquid to for segregating from the in specification bulk liquid. For automatic control of pumping and/or valving, any suitable process automation control can be employed, such as, but not limited to, a commercially available programmable logic controller (PLC), a PC, tablet, or other microprocessor based computing device, (sometimes collectively referred to herein by the term “control” or “controller”) and an associated user interface such as but not limited to, a touch screen/pad, monitor/keyboard, human machine interface (HMI), etc. A printer for printing out a ticket of the load can also be integrated. SCADA (supervisory control and data acquisition) or another suitable control network protocol can be enabled for remote data and command read/write capability. For mass and/or volume measurements, a conventional mass and/or volume flow meter or meters of suitable accuracy and real time data generation configured for incorporation in process piping can be utilized, and will be controlled by the controller and/or incorporate a transmitter for transmitting data to an associated controller and/or another location such as a remotely located control center, and optionally for receiving information and commands therefrom, including automated loading and unloading commands, if desired, for example, via conventional channels such as a wireless data connection and a secure VPN or similar well known data transfer arrangement. A non-limiting preferred type of meter suitable for the purposes of the invention are Coriolis mass flow meters commercially available from Krohne USA and other suppliers and Vorcone meters, typically operable to yield volume, density, mass flow, temperature, viscosity, pressure, velocity and volumetric flow rate of a fluid flow.

As a non-limiting example, a single meter can be employed for measuring properties of liquid being loaded and unloaded, or separate meters can be employed for measuring loading and unloading the liquid, respectively. As another possible configuration, two meters can be used in series. The meter or meters will preferably be disposed on the transport container, e.g., tanker trailer, truck, rail car or the like, or an associated vehicle such as a tractor truck, so as to travel from location to location therewith, as opposed to being permanently located at the collection tank, oil field, etc.

According to another preferred aspect of the invention, the meter or the associated controller is configured to determine substantially continuous values representative of density of the flow in real or near real time and compare those values to a value or values representative of at least one contaminant or impurity (herein sometimes collectively referred to by the term “contaminant”), such as, but not limited to, a non-conforming liquid such as water; and/or emulsions of water and/or oil, and/or solids, e.g., BS & W data broken down in specific detail, to detect presence thereof and for detailed analysis. Upon detection, the controller is operable to automatically perform a function that can be preset or selectable, including: to store and/or compile data representative thereof, e.g., a stream of density values, averages, running totals, etc., and occurrence in the flow, volume, or mass, e.g., by determining in discrete, predetermined segments of the flow such as by periodic sampling); communicate the presence to an associated signal or output device; transmit it to a remote device; and/or perform a designated operation or function, e.g., halt, reverse, or divert the flow to another location, such as a separate designated compartment of a transport container or another container. The controller can also automatically cease the flow and output a signal and await a command. As a non-limiting example, if presence of water of a certain quantity or characteristic is detected, it can be automatically diverted to a separate compartment of the tanker trailer or other transport container or another holding location, and if sufficient volume or mass is present, the load can be automatically or manually rejected and optionally returned to the collecting tank or other source. In addition or as an alternative, a signal or message can be transmitted to notify the owner and/or purchaser if a custody transfer is involved. If desired, the same or similar steps can be performed for another contaminant or impurity. Thus, for example, a transport tanker could have a compartment dedicated to receive water contaminants; and a separate compartment dedicated to receive solids contaminants, with the remaining compartment or compartments dedicated to receive in specification crude oil, and data registers can be provide to compile the contents of each by a designated parameter or parameters, such as density values determined from the measured masses of the flow, associated temperature values, etc. Running totals of amount of the liquid loaded or transferred, and average contaminant content can be computed and compiled, and stored, displayed, etc. Temperature can be continuously monitored, for example as an included meter function, or as separately sensed, and correlated with collected density values, and can be used to correct the values to a standard temperature, such as the 60 degrees Fahrenheit standard temperature used by the American Petroleum Institute or API, using a suitable programmed routine.

The collected data has many uses. In addition to precisely determining grade and individual contaminant levels, it can be compiled and tracked to enable monitoring well site equipment health such as dehydrators, desalters, filters, separators, etc. and determine efficiency, calibration, service requirements, predict problems, and the like.

In further to the above, crude oil will have a range of densities, e.g., from less than 720 kg/m³ for light crudes, to over 1000 kg/m³ for the heaviest crudes that establish its grade. The density of crude oil will vary with temperature—decreasing with increasing temperature, whereas viscosity decreases with increasing temperature. Water will have a density of about 1000 kg/m³, slightly higher if brine. The viscosity of water will be about the same within a range of temperatures. The same is true for emulsions typically encountered in crude oil. Emulsions found in crude oil will have lower densities, generally lower than the crude oil contained in a load. Thus a representative density value for identifying presence of water could be some value representative of 950-1000 kg/m³; and a representative density value for identifying presence of solids could be some value representative of 650-700 kg/m³, these obviously note being absolute values and being application sensitive.

Additional preferred hardware aspects of the invention can include an onboard panel, box, or other structure that carries the PLC or other controller, microprocessor, etc., a suitable power supply, communication device or devices such as, but not limited to, a wireless radio, network controller or router, modem, cellular modem, etc. for communicating with peripheral devices such as a PC, tablet or smart device, e.g., for enabling SCADA. As a non-limiting example, the PLC or other controller can communicate through a wiring harness, cables, etc., of an on-board network or wirelessly, e.g., WAN, with the operator interface and Coriolis meter, Vorcone meter, or other measuring device, and can receive inputs from and display information on an associated touch screen or the main operator interface device. The PLC or other controller can connect to a pump motor controller, valve controllers, such as but not limited to, pneumatic or electric servos, motors, solenoids, etc., for generating and controlling the liquid flow during loading and unloading, and also to signal devices, alarms, safety devices such as interlocks, etc., via a wiring harness, and/or a wired or wireless controller network or the like.

It can be recalled from the discussion above that loading crude oil for bulk transport from remote locations such as oil field collection and storage tanks, raises safety concerns when drivers have to climb tanks to make physical measurements of oil levels in the tanks; valuation issues when the crude oil is of a lower quality that expected; and integrity issues if the crude oil were to be skimmed or diluted during transport. By incorporating on-board flow rate measurement capability according to the invention, the volume of loaded liquid is automatically accurately measured, eliminating need for climbing tanks and measuring oil level, and the attendant dangers and possible inaccuracies and subjective errors.

Other representative user interface selections can include geographical location, address, well number, well owner, particular collection or storage tank, etc., where the load is to be loaded; volume of liquid to be loaded; whether valving is to be automatically or manually controlled by the operator/driver, etc. on site; whether the operator/driver is to be signaled/prompted when a compartment is full or filled to a specified amount; and whether out of specification liquid is present and/or segregated.

As a non-limiting example operator interface according to the invention, the operator or user, e.g., driver, is prompted by a suitable input output device, to choose from a list of stored sites where fluid can be loaded or unloaded.
All data can be entered into system by driver.
   All data is stored locally and transferred to a server through a secure network when connection is established.
   Driver can choose to load manually or in auto mode, or unload manually or in auto mode
      Manual mode—driver controls pump and valves for loading or unloading compartments
      Auto mode—driver selects how many compartments the trailer has, enters how many barrels or other units of measure of fluid is to be loaded into each compartment or unloaded, starts process and trailer loads or unloads automatically. In auto mode, driver still has master control.
   System can break total volume or mass down into water, oil and sediment via density. (BS&W)
   System notifies the driver if load has more than a selected or predetermined amount, e.g., 0.1%, water and/or other contaminants in load (crude oil application). This amount can be by mass or volume. Presence of contaminant or contaminants can be determined by determining density of flow with threshold values set for identifying particular contaminant, and running totals and averages can be compiled and outputted and/or displayed in real time or near real time.
   System will yield Gross Observed Volume, Gross Standard Volume and Net Standard Volume, of in specification load and contaminants individually, both for loading and unloading.
   System will communicate e.g., e-mail, customer directly after load has been loaded or unloaded with all pertinent data.
   System will track via GPS load from origin to destination.

System will give user a search function that will allow them to look up data from any previous run. If contaminant level or levels exceed set amount per unit of volume or mass, alarm (visual and/or audible, outputted and remote location can be notified. Driver or other operator (local or remote) or System automatically can determine next step
Interrupt loading or unloading.
Divert flow to alternative compartment or container designated for contaminant.
If contaminant level falls below set amount, divert loading back to designated compartment or container for in specification load.
Optional-pump collected contaminant(s) back into originating or source container.
Optional continue loading and after completion and settling of contaminant(s) pump back into originating or source container. Returned amount can be determined by volume or monitoring of flow density.

### BRIEF DESCRIPTION OF THE DRAWINGS

FIG. 1 is a side view of a bulk liquid tank trailer incorporating a Coriolis meter into piping thereof.

FIG. 1A is a top view of a Coriolis meter and piping of a bulk liquid tank trailer.

FIG. 1B is a side view of a Coriolis meter and piping of a bulk liquid tank trailer.

FIG. 1C is an image of a representative onside crude oil storage tank to which the bulk liquid tank trailer will be connected for loading crude for transport to another location.

FIG. 2 is a side view of a bulk liquid tank trailer incorporating two vorcone meters into piping thereof.

FIG. 3 is a side view of the tank trailer showing a control for loading and unloading, including for controlling the respective Coriolis or vorcone meter or meters and outputting data for the loading and unloading process.

FIG. 4 is an image of a user interface, illustrating an operating step of a method of the invention.

FIG. 5 is an image of a user interface, illustrating another operating step of a method according to the invention.

FIG. 6 is an image of a user interface, illustrating another operating step of a method according to the invention.

FIG. 7 is an image of a user interface, illustrating another operating step of a method according to the invention.

FIG. 8 is an image of a user interface, illustrating another operating step of a method according to the invention.

FIG. 8A is an image of a user interface, illustrating another operating step of a method according to the invention

FIG. 9 is an image of a user interface, illustrating another operating step of a method according to the invention.

FIG. 10 is an image of a user interface, illustrating another operating step of a method according to the invention.

FIG. 11 is an image of a user interface, illustrating another operating step of a method according to the invention.

FIG. 12 is a graphical representation of BS&W verses time determined for a representative loading operation according to the invention.

FIG. 13 is a graphical representation of flow rate verses volume determined for a representative loading operation according to the invention.

FIG. 14 is a tabular representation of sums of parameters including total volume loaded, BS&W, and total API, determined for a representative loading operation according to the invention.

### DETAILED DESCRIPTION OF PREFERRED EMBODIMENTS OF THE INVENTION

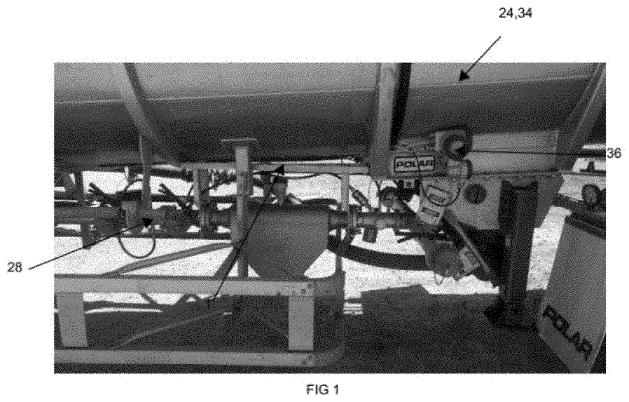

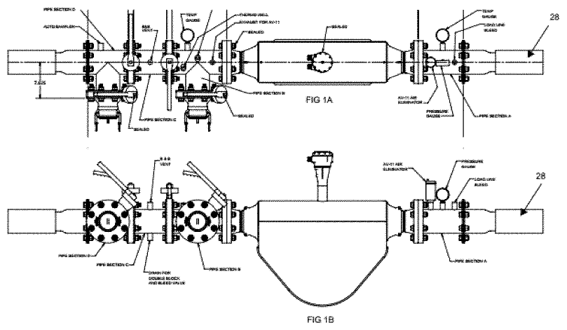

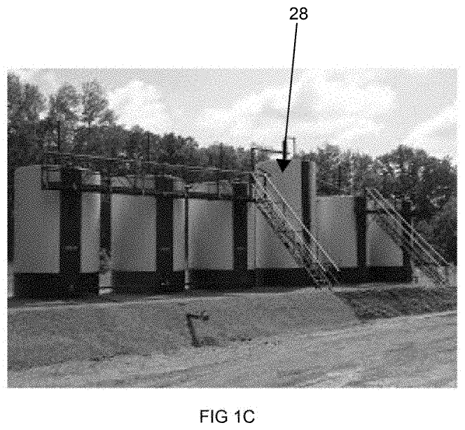

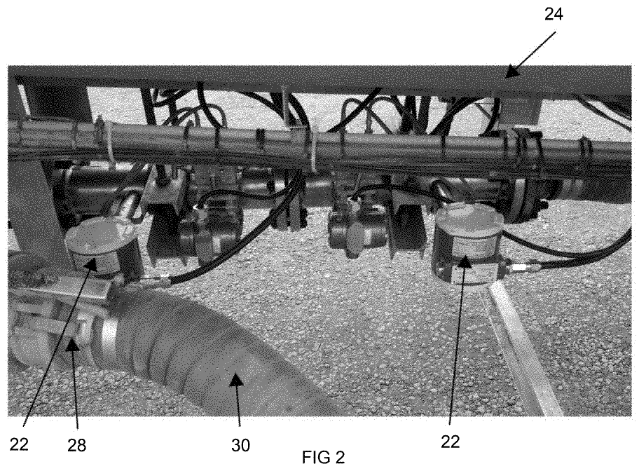

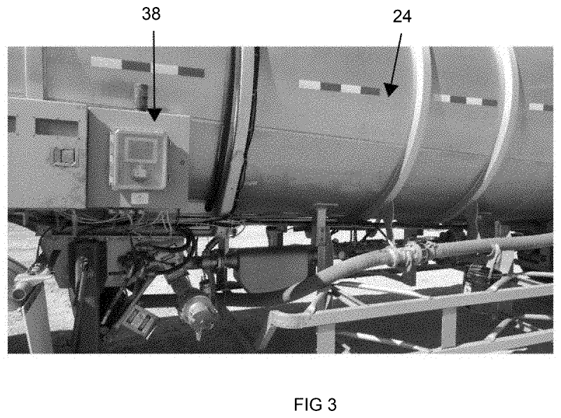

Referring now to the FIGS. 1, 1A, 1B, 2, and 3 of the drawings, the invention utilizes a meter 1 (FIGS. 1, 1A, 1B) or 22 (FIG. 2) or other measuring device or devices, configured and operable to determine mass, volume, and density of a flowing liquid, incorporated in a mobile platform for carrying on a bulk liquid or fluid transport vehicle 24 such as, but not limited to, a tanker trailer, tanker truck, rail tank car, or the like, having a typical onboard tank 34. As a non-limiting example, the meter 1 can comprise a Coriolis meter (FIGS. 1, 1A, 1B). The measurement of the mass flow rate in a Coriolis meter 1 is based on the principle of causing a medium to flow through a flow tube inserted in the pipe and vibrating during operation, whereby the medium is subjected to Coriolis forces. The latter causes the inlet-side and outlet-side portions of the flow tube to vibrate out of phase with respect to each other. The magnitude of these phase differences is a measure of the mass flow rate. The vibrations of the flow tube are therefore sensed by use of two vibration sensors positioned at a given distance from each other along the flow tube and converted by these sensors into measurement signals having a phase difference from which the mass flow rate is derived. A suitable commercially available Coriolis meter 1 for crude oil applications is the Optimass 6000 meter available from Krohne USA and configure for 4 inch piping connections.

Another suitable meter is a Vorcone meter 22 (FIG. 2) which is a hybrid vortex shedding and cone meter. In this type of meter fluid passing around a bluff body produces a stream of vortices with a generation rate which is proportional to the flow rate of the fluid. A sensor responsive to the vortices produces a signal having a frequency representing the flow rate. The flow rate signal can then be used for calculating the resulting volumetric flow rate of the fluid in the pipe. The measure of fluid flow rate for the vortex shedding flow meter, however, is independent of density. Thus, it is not possible to derive density or mass flow rate from the volumetric flow rate measurement alone. Therefore, an averaging pitot tube and a thermal flow meter, however, both measure flow rate dependent upon fluid density. A suitable Vorcone meter 22 is available from Vortek Instruments. A single meter 1, 22 can be utilized for loading and unloading with appropriate directional piping and valving, or two meters 1, 22 can be used.

Representative associated apparatus, namely, an onboard piping system 28, for incorporating the meter or meters 1, 22, in connection with an onboard tank 34 are generally illustrated. As illustrated in FIGS. 1A and 1B, the associated piping system 28 includes one or more thermometers 16; air eliminators 19, and pressure gages 20, incorporated onto a bulk liquid or fluid transport vehicle 24, and will variously include, but is not limited to: connecting flanges 2; gaskets 3; pipe 4, 11; hoses 8; bolts 5, 12, 13, nuts 6, 14, 17, and washers 18; and valves 7, 10, beneath tank 34 of the vehicle 24. Transport vehicle 24 shown here is configured to be utilized to unload crude from onsite storage and collection tanks such as tank 32 illustrated in FIG. 1C, which are commonly located at well sites in oil fields that can be located anywhere around the world. A typical transport vehicle 24 has a tank divided into 2 compartments, a front compartment and a rear compartment, and piping system 28 has automatically controllable valves in connection with each compartment so as to be configurable for directing flow of liquid to either or both of the compartments. Piling system 28 is additionally of sufficient length from the meter or meters 1, 22 to the compartments such that a segment or portion of the liquid flow that passes through the meter 1, 22 can be directed to a particular compartment after passing through and being measured by the meter. Piping system 28 will be connected to a coupler fitting of tank 32 via a hose 30 (FIG. 2) using standard couplers in the well known conventional manner. Piping system 28 or tank 32 can optionally include a pump for pumping the liquid from the tank 32 through hose 30 to the piping system 28 to tank 34, and/or gravity feed can be used. In this latter regard, it can be observed in FIG. 1C that onsite tank 32 is relatively substantial in height (at least twice as tall as vehicle 24) and thus when full or nearly full can generate a relatively high head pressure from gravity for initiating and maintaining the flow of the liquid into tank 34 of vehicle 24. A pump, either onsite or on board can be utilized when insufficient head pressure is present, or to supplement gravity for faster loading as desired or required for a particular application.

Additionally, the apparatus of the invention can include a H₂S detector 36 as shown in connection with vent piping of the container or tank 34, connected in a suitable conventional manner with the load unload control 38 so as to monitor H₂S emissions and generate a signal or alarm when present above a settable threshold level. The amount and timing of H₂S flow can also be recorded.

Control 38 includes an onboard panel, box, or other structure that carries a PLC or other microprocessor based controller, a suitable power supply, and a communication device or devices, which can be, for instance, a wireless radio, network controller or router, modem, cellular modem, etc. for communicating with peripheral devices such as a PC, tablet or smart device, e.g., for enabling SCADA and to provide a local or remote operator interface. The PLC or other controller communicates through a wiring harness, cables, etc., of an on-board network or wirelessly, e.g., WAN, with the operator interface and Coriolis meter, vorcone meter, or other measuring device, and receives inputs from and display information on an associated touch screen or the main operator interface device. The PLC or other controller connects to a pump motor controller, valve controllers, such as but not limited to, pneumatic or electric servos, motors, solenoids, etc., for generating and controlling the liquid flow during loading and unloading, to and from the compartments of vehicle 24, and also to signal devices, alarms, safety devices such as interlocks, etc., via a wiring harness, and/or a wired or wireless controller network or the like.

As discussed above, for crude oil loading applications it is often highly desirable to generate information and data regarding the oil being loaded, in particular, to precisely determine grade and individual contaminant levels, at the loading site, and/or when unloaded from the transport container at a destination such as an oil depot, pipeline terminal, or the like.

The apparatus, system, and method of the invention provide these capabilities, incorporated into an automatic loading routine that can be initiated when hose 32 of a transport vehicle 24 is securely coupled to a tank to be unloaded, such as tank 32.

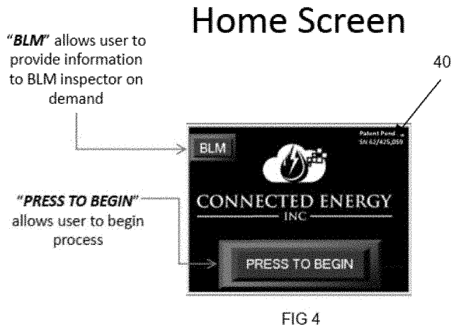

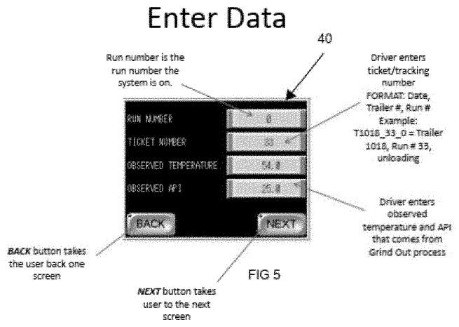

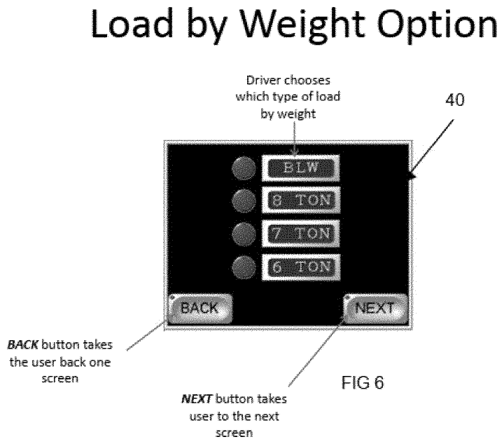

Referring also to FIGS. 4-11, a typical unloading sequence is initiated using an operator interface 40 connected to control 38, which operator interface 40 can be a touch screen, tablet, laptop, etc. or the main operator interface device (HMI) on control 38 itself. As a first step illustrated in FIG. 4, an operator initiates operation by touching “PRESS TO BEGIN”. Optionally, the operator can touch “BLM” to provide information to the Bureau of land management of the United States federal government. The next screen, shown in FIG. 5, will be an enter data screen, wherein the operator can enter a run number, which will be a number assigned to the particular load being loaded, and a ticket number for tracking purposes. An optional observed temperature taken from a thermometer measurement of a sample of the oil to be loaded is inputted as well as optionally an observed API value. The observed API value can be determined using an API measuring device and the sample of the oil to be loaded, in the well known manner. These are advantageous as they provide convenient references for values determined according to the invention. FIG. 6 shows a screen that is displayed to enable the operator to select a weight of the load to be loaded.

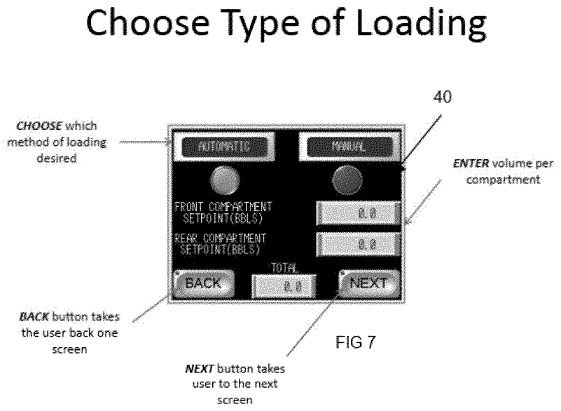

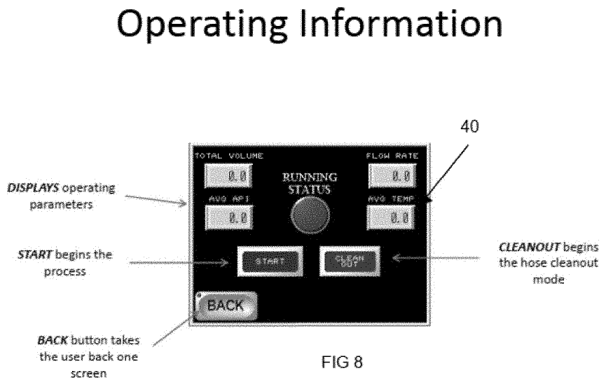

Referring to FIG. 7, the operator is next prompted to select a method of loading, either automatic or manual. If the vehicle transport container, e.g., vehicle 24, has 2 compartments, which is typical for transporting oil, the operator will be prompted to enter a set point value for the target volume for each compartment, at which loading will be automatically ceased by control 38.

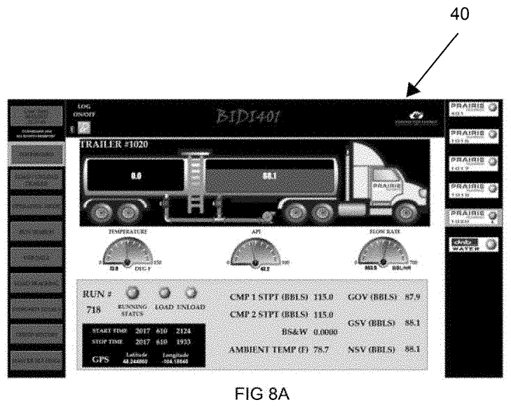

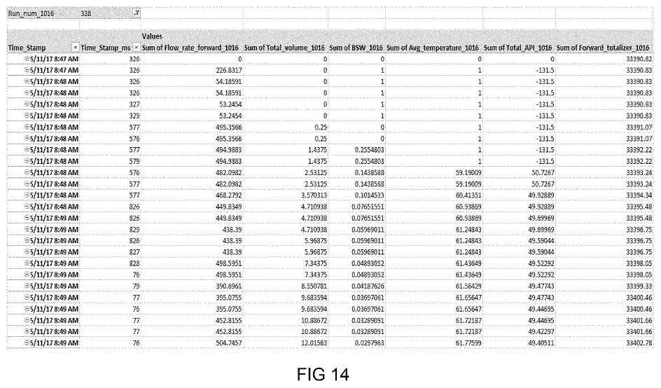

Referring now to FIG. 8, the operator is prompted to commence loading by pressing a “START” button. When the START button is pressed, the system opens the appropriate compartment valve of piping system 28, allowing flow into the selected compartment of the transport container. As the system begins the loading operation, control 38 will automatically determine total volume; average API; flow rate; and average temperature, during the loading operation and will compile those values and display them, continually updated, FIG. 14 is a table showing that data as compiled in the background. FIG. 8A is a graphical user interface (HMI) including graphical representations of load levels within the respective compartments of the transport container, and pertinent data determined and compiled during the loading operation according to the invention, including temperature, API, flow rate, gross observed volume (in barrels), gross standard volume (in barrels), net standard volume (in barrels), as well as percentage of BS&W (in barrels). This data is also compiled in the background as illustrated by FIG. 14.

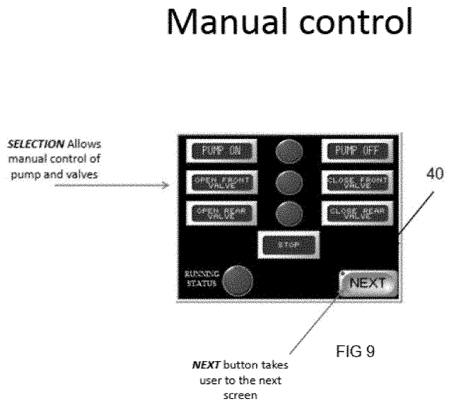

FIG. 9 is a user interface screen for optional manual control to enable the operator to manually operate the front and rear compartment valves and the pump.

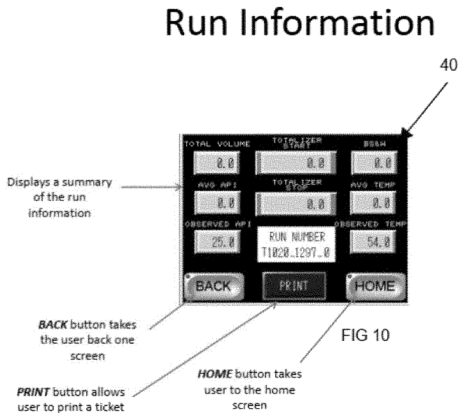

FIG. 10 is a user interface screen showing run information determined according to the invention. This information is computed and compiled automatically according to the invention and will include total volume, BS&W, average API, average temperature, and observed API and temperature from the initiation of the loading operation for reference. In this regard, if the average API determined by the invention differs significantly from observed API, the operator is alerted of a possible problem and can investigate, report, and/or correct any problems discovered.

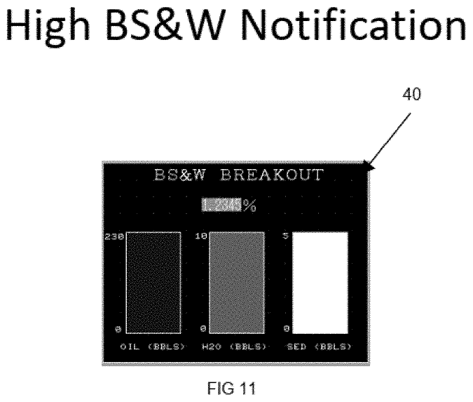

In FIG. 11, a screen is shown including a high BS&W notification output capability of the system of the invention. The system has the capability for allowing the operator or other supervisory personnel to enter a threshold value for BS&W for the load being loaded. This value can be expressed as a percentage of the load or volume, or both. As an exemplary option, the system will automatically display a total value for barrels of oil loaded, and barrels of water and sediment (solids). In the background, the system automatically monitors running BS&W values determined from determinations of density of segments of the liquid of the flow, and compares those values to a set threshold value. A time threshold for BS&W out of limit can be set. The system can also be set to automatically output an audible and/or visual signal, such as an audible alarm and/or signal light, in the event the threshold value is exceeded, at any time, or for the set time period.

The system can also perform an automatic operation to return or segregate the high BS&W liquid. As an example, the quantity of BS&W in a tank to be unloaded is typically greatest at the bottom, which is the portion of the tank typically unloaded first. Whichever compartment of the transport container selected to be loaded first, that compartment will receive the initial BS&W from the bottom of the tank being unloaded. Subsequently, during the loading operation there may be little BS&W. However, that may not be the case. For instance, trapped or captured water or solids may be present elsewhere in a tank, or the bottom of a second tank may be loaded, so as to introduce more BS&W into the load to be transported.

If the above scenarios occur, and the incoming BS&W exceeds a set threshold value, the system can be programmed to automatically divert that flow to the other compartment designated for receiving BS&W. Typically, transport tanks are filled from the bottom, and therefore the BS&W will have a tendency to be located in the bottom region of the designated compartment. Now, if desired, that region of the designated compartment can be separately unloaded, including by being pumped back into the tank being unloaded if desired, so that the load to be transported will have higher quality, or at least be segregated, if it is elected to not pump back the BS&W. As noted above, the above described metrics of the load can be stored by the system of the invention, as well as outputted to a desired destination, such as supervisory personnel and/or customers, or the like.

As another scenario of operation that can be employed, the BS&W will tend to settle into the receiving compartment or container during loading, and after loading the system can be programmed to automatically remove a designated portion of the contents containing a higher concentration of the BS&W and return it to the sending container or direct it to another location. Because the apparatus and system of the invention determines BS&W in during the flow, that information can be determined during the removal and the removal flow can be automatically terminated when a set threshold value, e.g., percentage or concentration in the return flow, is reached. Thus, lower quality crude containing a higher percentage of BS&W can be automatically separated and segregated from the higher quality, if desired.

The meters 1, 22, as explained above are each operable to determine values representative of the density of the liquid flowing therethrough. Essentially, the sensing apparatus and data processing capabilities of the apparatus and processor enable the densities to be accurately determined for a portion or segment of the flow of the liquid, at very short time intervals, e.g., a few hundred milliseconds, which, for purposes of the invention can be expressed as segments or slices of the flow of the liquid through the meter 1 or 22. Solids are known to have a range of density values (typically expressed in kilograms/liter) that are less than a threshold value that will be less than the density values of the vast majority of grades of oil found in crude; water is known to have a range of density values greater than a threshold value greater than the density values for the pertinent grades of oil. Thus, the invention determines the densities for the segments of the flow on a time segment basis, on a continuing or ongoing basis, and compares the determined density values to a lower threshold value that will identify it as a solid, e.g., set between 0.64 and 0.70 kg/m³ for oil extracted from the Bakken fields of the US, and compares the density values to a higher threshold value that will identify it as water, e.g., set between 0.9 and 0.94 kg/m³ for Bakken oil, those segments that have densities between the threshold values will be identified as oil. Running totals of each category of density are then compiled. For example, because flow rate is also being measured, the categories are correlated to flow and compiled in barrels per some time period, e.g., per second, of flow.

It is desired to determine an average API value for the liquid periodically during the loading operation. API is a dimensionless value and can be calculated using the formulas set forth below. The term “Oil” represents a density value for oil as determined by the meter 1 or 22, and the term “Water” represents a density value for water as determined by the meter. Some government regulators require average API values to be recorded periodically for a load, and this is intended to comply with this requirement. The system of the invention averages the compiled density values for oil and water over predetermined intervals, here, 10 second time intervals, although it should be recognized that shorter or longer time intervals can be utilized. These average oil and water density values are then used to calculate average API for each of the predetermined intervals, on a continuous basis during the flow. The density averages are correlated for temperature for determining standard values. These average API values are then displayed on a running basis on the operator interface with associated average temperature values.

API=141.5/(Oil/Water)−131.5

API=141.5*(Oil/Water)−131.5

Oil*API=(141.5*Water/Oil)*Oil−131.5*Oil

Oil*API+131.5*Oil−141.5*Water−131.5*Oil+131.5*Oil

Oil*API+131.5*Oil=141.5*Water

OIl*(API+131.5)=141.5*Water

Oil*(API+131.5)/(API+131.5)=(141.5*Water)/(API+131.5)

Oil=(141.5*Water)/(API+131.5)

[Observed API−0.059175*(Observed Temp−60)]/[1+0.00045*(Observed Temp−60)]
Observed Temp is in F

An exemplary method of loading a liquid from a first container into a second container according to the invention can comprise steps of:

generating a flow of the liquid through a conduit from the first container toward the second container while automatically

monitoring characteristics of sequential predetermined segments of the liquid of the flow or for a predetermined time segment of the flow, and determining values representative of densities of the predetermined segments of the flow, respectively;

comparing the values representative of the densities of each of the predetermined segments of the flow or the predetermined time period of the flow to at least one predetermined value to determine presence of at least one contaminant therein, respectively, and:

compiling a first running total of the values representative of the densities of the predetermined segments of the liquid of the flow or the predetermined time period of the flow determined to lack the presence of the at least one contaminant therein; and;

compiling a second running total of the values representative of the densities of the predetermined segments of the liquid of the flow or the predetermined time period of the flow determined to have the at least one contaminant therein.

Another exemplary method of loading a liquid from at least one stationary collection container proximate a production source of the liquid, into a bulk liquid transport container for transport of the liquid to another location, can comprise steps of:

generating an initial flow of the liquid through a conduit from the collection container toward the transport container;

and automatically

monitoring characteristics of the initial flow and determining at least one initial density value for the initial flow therefrom;

comparing the at least one initial density value for the initial flow to a value indicative of presence of a contaminant, and:

i. if the comparison is indicative of presence of the contaminant, then performing at least one of the following steps:
a. outputting a signal;
b. ceasing the loading; and
c. returning the initial flow to the collection container or transferring the initial flow to another container;
   and

ii. if the comparison is indicative of absence of the contaminant, then continuing the flow and the steps of monitoring and comparing, until either:
a. expiration of a predetermined period of time;
b. a predetermined amount of the liquid has been loaded, or

the liquid flow is absent for a predetermined period of time.

Still another method according to the invention of loading crude oil from at least one stationary collection container proximate a production source of the crude oil, into a bulk liquid transport container for transport of the crude oil to another location, comprises steps of:

providing a first value indicative of presence of a contaminant in the crude oil;

generating an initial flow of the crude oil through a conduit from the collection container toward the transport container;

and automatically

monitoring characteristics of the initial loading flow and determining at least one initial density value for the flow therefrom;

comparing the at least one initial density value to the value indicative of presence of a contaminant, and:

iii. if the comparison is indicative of presence of the contaminant, then performing at least one of the following steps:
d. outputting a signal;
e. ceasing the loading; and
f. returning the initial flow to the collection container;
   and

iv. if the comparison is indicative of absence of the contaminant, then continuing the flow and the steps of monitoring and comparing, until either:
c. expiration of a predetermined period of time;
d. a predetermined amount of the crude oil has been loaded, or

the flow is absent for a predetermined period of time.

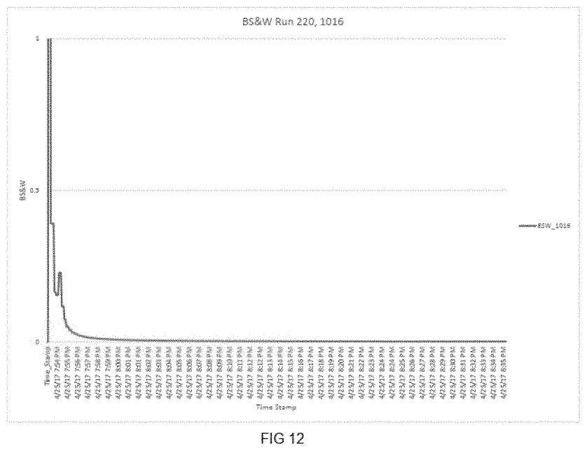

FIG. 12 is a graphical representation of BS&W verses time determined according to the invention for a representative loading operation wherein a typical transport vehicle such as vehicle 24 described herein is loaded from a storage tank such as a tank 32. The BS&W is expressed as a percentage of the load as it is being loaded. Thus, as can be expected, the percentage BS&W is initially high due to settling of the water and sediments in the bottom of the tank being unloaded, the bottom being unloaded first. The percentage of BS&W rapidly tapers off to almost zero. This BS&W percentage is determined from the density values for the segments of the liquid of the flow outputted by the meter 1 or 22 and associated with a volume value (in barrels) derived from the flow rate. That is, since each determined density value represents a segment of time of the flow of some designated number of milliseconds, and the flow rate is known for that segment of time, the volume of the liquid at that density is calculated. In the graph of FIG. 12, the intervals displayed are 1 minute, so the volumes of segments of the flow identified by density as oil, and those identified as solids and water combined, are determined, and the percentage of the total comprising the BS&W displayed as shown. Because the BS&W is determined by control 38 on board vehicle 24 essentially in real time or near real time, if a BS&W value greater than a set value as a density or a percentage of total is detected, a signal or alarm can be automatically outputted and/or a predetermined action automatically taken, e.g., shut down flow, divert flow to a different location, e.g., other compartment, or separate location. In this manner, the BS&W over a set limit for the load can be segregated into a designated compartment, and can be offloaded in a special manner to preserve the remainder of the load at a lower BS&W level and thus higher quality. This graphical representation illustrates the advantage of more accurate data collection achieved by the continuous determining of the BS&W percentage during the entire loading operation, compared to presently used methods of industry wherein BS&W content can be measured from liquid samples are taken manually at intervals such as ¼. ½/and ¾ through the loading operation.

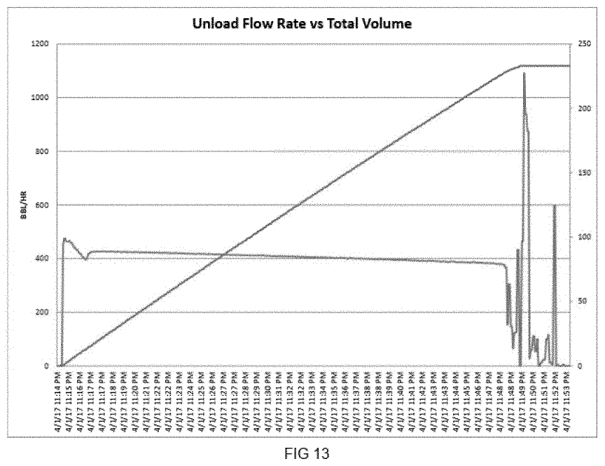

FIG. 13 is a graphical representation of flow rate and total volume (in barrels) verses time in one minute intervals determined by the system of the invention for a representative loading operation.

FIG. 14 is a table compiling flow rate in barrels per hour, total volume loaded in barrels, BS&W as a percentage of the volume, average temperature, API, an a volume totalizer value, all verses time at predetermined millisecond intervals, determined by the invention for a portion of another representative loading operation. Here, it can be observed that initial default values 1 and 0 are used for BS&W and temperature during the initial loading when the piping system is not yet filled with the liquid being loaded. The system can be programmed to ignore those values in the computations for the load so as to produce more accuracy.

In light of all the foregoing, it should thus be apparent to those skilled in the art that there has been shown and described an ONBOARD APPARATUS, SYSTEM, AND METHOD FOR AUTOMATICALLY DYNAMICALLY EVALUATING CHARACTERISTICS OF A NON-HOMOGENOUS LIQUID DURING LOADING AND UNLOADING OF A TRANSPORT CONTAINER. However, it should also be apparent that, within the principles and scope of the invention, many changes are possible and contemplated, including in the details, materials, and arrangements of parts which have been described and illustrated to explain the nature of the invention. Thus, while the foregoing description and discussion addresses certain preferred embodiments or elements of the invention, it should further be understood that concepts of the invention, as based upon the foregoing description and discussion, may be readily incorporated into or employed in other embodiments and constructions without departing from the scope of the invention. Accordingly, the following claims are intended to protect the invention broadly as well as in the specific form shown, and all changes, modifications, variations, and other uses and applications which do not depart from the spirit and scope of the invention are deemed to be covered by the invention, which is limited only by the claims which follow.

What is claimed is:

1. A method of loading a liquid from a first container into a second container, comprising steps of:
generating a flow of the liquid through a conduit from the first container toward the second container while automatically
monitoring characteristics of sequential predetermined segments of the liquid of the flow or for a predetermined time segment of the flow, and determining values representative of densities of the predetermined segments of the flow, respectively;
comparing the values representative of the densities of each of the predetermined segments of the flow or the predetermined time period of the flow to at least one predetermined value to determine presence of at least one contaminant therein, respectively, and:
a. compiling a first running total of the values representative of the densities of the predetermined segments of the liquid of the flow or the predetermined time period of the flow determined to lack the presence of the at least one contaminant therein; and;
e. compiling a second running total of the values representative of the densities of the predetermined segments of the liquid of the flow or the predetermined have the at least one contaminant therein.

2. The method of claim 1, comprising a step of determining an average of the values representative of the densities of the predetermined segments of the liquid of the flow or the predetermined time period of the flow determined to have the at least one contaminant therein.

3. The method of claim 2, comprising an additional step of monitoring the determined average of the values representative of the densities of the predetermined segments of the liquid of the flow or the predetermined time period of the flow determined to have the at least one contaminant therein, then performing at least one of the following steps:
g. outputting a signal indicative thereof; and
b. ceasing the loading if the determined average exceeds a predetermined threshold value.

4. The method of claim 1, comprising an additional step of determining a running total for the loaded liquid as a function of the compiled first and second running totals.

5. The method of claim 1, comprising a step of communicating at least one of the compiled first and second running totals to at least one recipient or potential recipient for the liquid.

6. The method of claim 1, wherein apparatus for performing the step of monitoring the characteristics of the sequential predetermined segments of the liquid of the flow or the predetermined time period of the flow are located on a bulk liquid transport vehicle comprising one of the first container or the second container.

7. The method of claim 1, wherein the values representative of densities of the predetermined segments of the liquid of the flow or the predetermined time period of the flow are compared to a predetermined value representative of air contained in the segment to exclude the values representative of densities of the predetermined segments of the flow found to contain air from at least the second running total.

8. The method of claim 1, wherein the second container is a transport container, and the method comprising further steps of:
providing at least two separate compartments within the transport container and connected to the conduit, respectively; and
automatically
directing an initial portion of the flow of the liquid to a first of the compartments, then, after the loading, returning the initial portion of the flow of the liquid to the first container or transferring the initial portion of the flow to another container.

9. The method of claim 1, wherein at least one of the containers is a transport container, and a vehicle connected thereto comprises instruments including at least a density meter and a thermometer, configured to automatically monitor the characteristics of the flow of the liquid and determine the density values and an associated temperature, and a processor connected thereto, configured and operable to perform the comparing step.

10. The method of claim 1, wherein the liquid comprises crude oil and the contaminant comprises water.

11. The method of claim 1, wherein the liquid comprises crude oil and the contaminant comprises solids.

12. The method of claim 1, wherein the liquid comprises crude oil and the contaminant comprises an emulsion including solids.

13. The method of claim 1, wherein the liquid comprises crude oil and the first container comprises a stationary tank in an oil field or in close proximity thereto.

14. A method of loading a liquid from at least one stationary collection container proximate a production source of the liquid, into a bulk liquid transport container for transport of the liquid to another location, comprising steps of:
generating an initial flow of the liquid through a conduit from the collection container toward the transport container;
and automatically
monitoring characteristics of the initial flow and determining at least one initial density value for the initial flow therefrom;
comparing the at least one initial density value for the initial flow to a value indicative of presence of a contaminant, and:
v. if the comparison is indicative of presence of the contaminant, then performing at least one of the following steps:
h. outputting a signal;
i. ceasing the loading; and
j. returning the initial flow to the collection container or transferring the initial flow to another container;
and
vi. if the comparison is indicative of absence of the contaminant, then continuing the flow and the steps of monitoring and comparing, until either:
f. expiration of a predetermined period of time;
g. a predetermined amount of the liquid has been loaded, or
h. the liquid flow is absent for a predetermined period of time.

15. The method of claim 14, where in step ii. the step of comparing involves determining the presence of the contaminant, and if present, then performing at least one of the following steps:
a. outputting a signal indicative thereof;
b. ceasing the loading.

16. The method of claim 15, comprising an additional step of compiling the density values for at least the continuing flow in a data file associated with the loaded liquid.

17. The method of claim 16, comprising an additional step of determining a value for the loaded liquid as a function of at least the compiled density values.

18. The method of claim 16, comprising a step of communicating the compiled density values or the determined value for the loaded liquid to at least one recipient or potential recipient for the liquid.

19. The method of claim 14, wherein apparatus for performing the steps of monitoring are located on the bulk liquid transport container or a vehicle that moves therewith, and the method comprises further steps of:
unloading the loaded liquid from the transport container in an unloading flow;
and automatically
monitoring characteristics of the unloading flow and determining multiple density values therefor; and
comparing the multiple density values for the unloading flow to the density values for the continuing flow to verify integrity of the unloaded liquid.

20. The method of claim 19, wherein the comparing of the density values for the unloading flow and the continuing flow are used to calibrate the apparatus on the bulk liquid transport container or vehicle that moves therewith.

21. The method of claim 14, comprising further steps of:
providing at least two separate compartments within the transport container and connected to the conduit, respectively; and
automatically
directing the continuing flow to a first of the compartments, while monitoring characteristics thereof and periodically determining continuing density values therefrom, and comparing the continuing density values or at least one value representative thereof to at least one limit value; and
if the continuing density values or the at least one value representative thereof are or is beyond the limit, then diverting the flow to a second of the compartments.

22. The method of claim 21, comprising further steps of:
while diverting the flow, monitoring characteristics of the diverted flow and periodically determining diverted flow density values therefrom, and comparing the diverted flow density values or at least one representative value thereof to at least one predetermined limit therefor; and
if beyond the at least one predetermined limit, then diverting the flow to the first of the compartments.

22. The method of claim 20, comprising a further step after completion of the loading of the liquid into the transport container, of unloading at least a portion of any contents of the second compartment.

23. The method of claim 22, where in the step of unloading, the contents of the second compartment are automatically unloaded into the collection container upon completion of the loading.

24. The method of claim 14, wherein the transport container or a vehicle connected thereto comprises instruments including at least a density meter, and a thermometer, configured to automatically monitor the characteristics of the loading flow and determine the density values and an associated temperature, and a processor connected thereto, configured and operable to perform the comparing step.

25. The method of claim 14, wherein the liquid comprises crude oil and the contaminant comprises water.

26. The method of claim 14, wherein the value indicative of presence of a contaminant is at least about 0.9.

27. The method of claim 14, wherein the liquid comprises crude oil and the contaminant comprises an emulsion including solids.

28. The method of claim 14, wherein the value indicative of presence of a contaminant is less than about 0.7.

29. The method of claim 14, wherein step i. a. further comprises prompting a user to select at least one of step i. b. and step i. c.

30. The method of claim 14, comprising further steps of:
automatically:
moving the transport container to another location; then
unloading the liquid from the transport container into a different container, comprising steps of:
generating an unloading flow of the liquid through a conduit from the transport container to the different container; while
monitoring characteristics of the unloading flow, including determining multiple unloading flow density values thereof and comparing the unloading flow density values or at least one value representative thereof to the density values determined for the continuing flow or a value representative thereof.

31. The method of claim 30, wherein at least a substantial portion of the monitoring steps are performed by apparatus on the transport container or a vehicle that moves therewith.

32. The method of claim 30, wherein values obtained from the comparing of the unloading flow density values or at least one value representative thereof to the density values determined for the continuing flow or a value representative thereof are used to calibrate the apparatus on the transport container or vehicle that moves therewith.

33. The method of claim 14, wherein the transport container comprises a container selected from a group consisting of: a tanker truck, a tanker trailer, and a rail car tanker.

34. The method of claim 14, wherein the liquid comprises crude oil and the collection container comprises a stationary tank in an oil field or in close proximity thereto.

35. The method of claim 14, wherein the value indicative of presence of water comprises a value representative of a density value of at least about 0.9 kg/m³.

36. A method of loading crude oil from at least one stationary collection container proximate a production source of the crude oil, into a bulk liquid transport container for transport of the crude oil to another location, comprising steps of:
providing a first value indicative of presence of a contaminant in the crude oil;
generating an initial flow of the crude oil through a conduit from the collection container toward the transport container;
and automatically
monitoring characteristics of the initial loading flow and determining at least one initial density value for the flow therefrom;
comparing the at least one initial density value to the value indicative of presence of a contaminant, and:
vii. if the comparison is indicative of presence of the contaminant, then performing at least one of the following steps:
k. outputting a signal;
l. ceasing the loading; and
m. returning the initial flow to the collection container;
and
viii. if the comparison is indicative of absence of the contaminant, then continuing the flow and the steps of monitoring and comparing, until either:
i. expiration of a predetermined period of time;
j. a predetermined amount of the crude oil has been loaded, or
k. the flow is absent for a predetermined period of time.
